(19) United States
(12) Patent Application Publication
(10) Pub. No.: US 2006/0063834 A1
(43) Pub. Date: Mar. 23, 2006

(54) Substituted adamantanes, and methods of making the same

(76) Inventors: John V. Frangioni, Wayland, MA (US); Wolfgang Maison, Hamburg (DE); Daniel S. Kemp, Boston, MA (US)

Correspondence Address: FISH & RICHARDSON PC, P.O. BOX 1022, MINNEAPOLIS, MN, 55440-1022, US

(21) Appl. No.: 11/222,951

(22) Filed: Sep. 9, 2005

Related U.S. Application Data
(60) Provisional application No. 60/609,198, filed on Sep. 9, 2004.

(51) Int. Cl. A61K 31/275; A61K 31/16; C07C 255/47

(52) U.S. Cl. 514/519; 514/613; 558/429; 564/123

(57) ABSTRACT

Adamantane derivatives, and methods of making and using the same are disclosed.

### CROSS REFERENCE TO RELATED APPLICATIONS

This application claims priority from U.S. Provisional Patent Application No. 60/609,198, filed on Sep. 9, 2004, the contents of which is incorporated herein by reference in its entirety.

### STATEMENT AS TO FEDERALLY SPONSORED RESEARCH

This invention was made with Government support under NIH Grant Nos. R21 CA-88870, R21/33 CA-88245 and R21/R33 EB-00673; and under Department of Energy grant No. DE-FG02-01ER63188. Thus, the Government has certain rights in the invention.

### TECHNICAL FIELD

This invention relates to substituted adamantanes, and methods of making and using the same.

### BACKGROUND

Adamantane 1 is a rigid molecule that includes three fused, six-membered carbocyclic rings. Bridgehead carbon atoms are arranged to occupy positions 1, 3, 5 and 7. Derivatives of adamantane can be useful in medicine and in materials science. Uses in medicine are described, e.g., by Henkel, J. Med. Chem. 1982, 25, 5156 and Zah, Bioorg. Med. Chem. 2003, 11, 3569. Uses in materials science are described, e.g., by Li in J. Org. Chem. 2004, 69, 1010 and J. Org. Chem. 2003, 68, 4862, and Radhakrishnan in Org. Lett. 2001, 3, 3141.

### SUMMARY

Generally, adamantane derivatives described herein have a molecular arm extending from each bridgehead position of the adamantane nucleus. Generally, such molecules have a structure that can be represented by Structure I

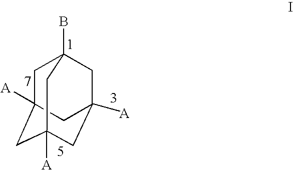

Such derivatives have at least two parts, e.g., part A and part B. Part A is a first moiety that includes a hydrocarbon having less than 6 carbon atoms and a carbon-carbon multiple bond; a carboxylic acid group, or an ester, anhydride, or acid halide thereof, and can include, e.g., a spacer portion (e.g., a rigid, hydrophilic spacer portion). A targeting portion or moiety can extend from an end of the spacer portion such that it can interact with complementary sites, e.g., on a cell. Part B is a second moiety that can, e.g., include a nucleophile or a protected nucleophile, or can include, e.g., either a contrast agent (for imaging), or a therapeutic agent, or both. Part B can optionally include a spacer.

When spacer portions are present, they can include, e.g., an α-helix, or a dimeric coil-coil. In some embodiments, the α-helix can be cross-linked, e.g., for enhanced rigidity. In certain embodiments, each spacer portion can have a molecular weight of from about 500 Daltons to about 300,000 Daltons. Each spacer portion can have, e.g., a length of from about 1 nm to about 100 nm. In some embodiments, each arm that includes a spacer portion, also includes a targeting moiety bonded to the spacer portion, e.g., a targeting moiety that is, or is, derived from a peptide or polypeptide selected, e.g., RGD peptide, melanocyte stimulating hormone (MSH), or somatostatin.

In one aspect, the invention features compounds including an adamantane core having four bridgehead positions, wherein one, two, or three of the four bridgehead positions are functionalized with a first moiety that includes a carboxylic acid group, or an ester, anhydride, or acid halide thereof. One or more, e.g., the fourth, bridgehead position includes a second moiety different from the first moiety. The second moiety can include a nucleophile or a protected nucleophile.

For example, the protecting group can be, e.g., t-butoxycarbonyl (Boc) or benzyloxycarbonyl. The nucleophile can be, e.g., an amino group. The carboxylic acid group, or an ester, anhydride, or acid halide thereof can be, e.g., at a terminal end of a hydrocarbon or substituted hydrocarbon chain, e.g., an unsaturated hydrocarbon. In some embodiments, the nucleophile can be at a terminal end of a hydrocarbon or substituted hydrocarbon chain.

The compounds can further include, e.g., a ligand bound to at least a portion of at least one of the first moieties. For example, a ligand can include a targeting compound, e.g., an RGD peptide, a melanocyte stimulating hormone (MSH), or somatostatin.

The compounds can further include, e.g., a reporter molecule bound to at least a portion of the second moiety.

In another aspect, the invention features compounds of Structure I (above, and in FIG. 1) in which B is H, F, Cl, Br, I, CN, an N-acetyl group, an ammonium group, an amino group, or a protected amino group. Each A is independently a moiety that includes a hydrocarbon having 6 or fewer carbon atoms and comprising at least one carbon-carbon multiple bond, a carboxylic acid group, or an ester, anhydride, or acid halide thereof.

In some embodiments, B is Br, NH₃Cl or HN(Boc), and each A is CH₂CH₂CO₂H, or an NHS ester thereof. In certain embodiments, B is a protected amino group, and each A is COOH, or an NHS ester thereof. In other embodiments, B is CN, and each A is the methyl ester of COOH.

It can be advantageous, in some embodiments (e.g., when it is desirable to have rigid arms), to have an A and/or a B in which there is hindered rotation about at least one bond of A and/or B. For example, A and/or B can include a carbon-carbon double bond, a carbon-carbon triple bond, an aryl group or a constrained ring system. It can also be advantageous, in some embodiments, that A contain less than 8 carbon atoms. For example, each A can be independently COOH or CH₂COOH, or a methyl ester or NHS ester thereof.

In some embodiments, at least one A includes a linking moiety, e.g., a hydrocarbon chain that includes a terminal carboxylic acid, ester, anhydride, acid halide, halogen, amino group, or hydroxyl group, or a substituted hydrocarbon chain that includes a terminal carboxylic acid, ester, anhydride, acid halide, halogen, amino group, or hydroxyl group.

In some embodiments, the compounds further include a ligand bound to at least a portion of each A. Each ligand can be the same or different. The ligand can include, e.g., a targeting compound, e.g., a peptide or polypeptide (e.g., RGD peptide, melanocyte stimulating hormone (MSH), or somatostatin). The targeting compound can be, e.g., further linked to a contrast agent.

In some embodiments, the compounds further include a reporter molecule bound to at least a portion of B.

In some embodiments, the compounds further include a therapeutic agent bound to at least a portion of B, e.g., doxorubicin, taxol, DOTA, a moiety that includes boron, or a pro-apoptotic peptide.

In a specific embodiment, the compound is compound 5 of FIG. 6.

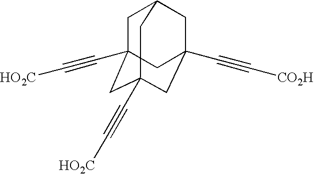

In certain embodiments, the at least one carbon-carbon multiple bond can be a carbon-carbon triple bond. In a specific embodiment, the compound has a structure represented by

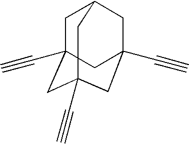

In another aspect, the invention features methods of making compounds of Structure I′ (FIG. 2), in which L₁, L₂ and L₃ are ligands that can be the same or different. 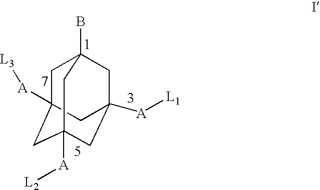
The methods include providing a compound of Structure I, and reacting the compound of Structure I with a ligand such that the ligand reacts selectively with A through the carboxylic acid, ester, anhydride, or acid halide of A.

In another aspect, the invention features methods of making compounds represented by Structure I″ (FIG. 3). Ligands L₁, L₂ and L₃ can be the same or different. 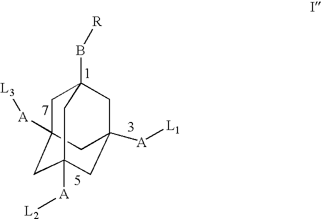
The methods include providing a compound of Structure I′, and reacting the compound of Structure I′ with a reporter group or therapeutic group (R) such that the reporter or therapeutic group reacts selectively with B.

The invention also features methods of making compounds represented by Structure I′″ (FIG. 4). 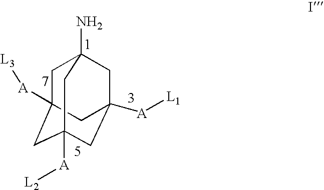
The methods include providing a compound of Structure I′ in which B includes an amino protecting group, and deprotecting the amino group.

The invention also features methods of making compounds represented by Structure I″″ (FIG. 5). 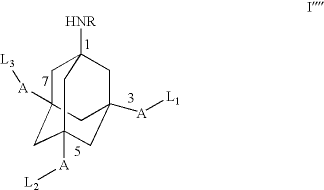
The methods include providing a compound of Structure I′″, and reacting the compound of Structure I′″ with a reporter or therapeutic group (R) such that the reporter or therapeutic group reacts selectively with the amino group.

In another aspect, the invention features methods of imaging a portion of an animal or human body. The methods include providing a compound of Structure I, I′, I″, I′″, or I″″ (FIGS. 1, 2, 3, 4, and 5, respectively) including a reporter group and a targeting ligand that specifically binds to a moiety in the portion of the body. The compound is administered to the body, and, after a sufficient time for the targeting ligand to selectively bind to the moiety in the portion, imaging the portion of the body.

The adamantane derivatives described herein can be used in medicine, e.g., as medical imaging agents when appropriately conjugated, as drugs, or as drug delivery systems. Specific applications include, e.g., in vivo tumor drug targeting and in-vivo tumor imaging. Many of the admantane derivatives are particularly efficient at bonding to complementary sites, e.g., on cell walls and can have, e.g., a long blood half life.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a structure showing adamantane derivatives functionalized at bridgehead carbon atoms with groups A and B.

FIG. 2 is a structure showing adamantane derivatives resulting from reaction of the adamantane derivatives shown in FIG. 1 with ligands.

FIG. 3 is a structure showing adamantane derivatives resulting from reaction of the adamantane derivatives shown in FIG. 2 with reporter molecules.

FIG. 4 is a structure showing adamantane derivatives after removal of a protecting group.

FIG. 5 is a structure showing adamantane derivatives resulting from the reaction of the adamantane derivatives of FIG. 4 with reporter molecules.

FIG. 6 is a reaction scheme illustrating the preparation of compound 10 starting from adamantane 1.

FIGS. 7-8 are reaction schemes illustrating the preparation of compound 14 starting from adamantane 1.

FIGS. 9-10 are reaction schemes illustrating the preparation of compound 19 starting from a bromoadamantane 15.

### DETAILED DESCRIPTION

Some of the adamantane derivatives described herein have a molecular arm that extends from each bridgehead positions of the adamantane nucleus, i.e., at carbons 1, 3, 5 and 7 of the adamantane nucleus. Generally, the arms extending from carbons 3, 5, and 7 can be used for attachment of ligands (e.g., targeting ligands), and/or linking or spacer moieties, and a fourth arm extending from the carbon 1 position can be used for conjugation to other molecules, e.g., therapeutic agent or imaging agents.

Many of the adamantane derivatives described herein have a low entropy in that they are mechanically rigid, e.g., have hindered rotation about bonds, and conformationally well defined. Some of the admantane derivatives are also hydrophilic or include portions which are hydrophilic.

Other adamantane derivatives are provided that include two parts that can be attached to various other molecules. For example, targeting ligands that interact with complementary receptor sites on cells, can be attached to a first part, and a contrast agent, or a therapeutic agent can be attached to a second part.

Base Adamantane Derivatives

Some base adamantane derivatives include an adamantane core having four bridgehead positions. Three of the four bridgehead positions are functionalized with a first moiety that includes a carboxylic acid group, or an ester, anhydride, or acid halide thereof. The fourth bridgehead position includes a second moiety different from the first moiety. The second moiety includes a nucleophile, e.g., an amino group, or a protected nucleophile, e.g., protected amino group.

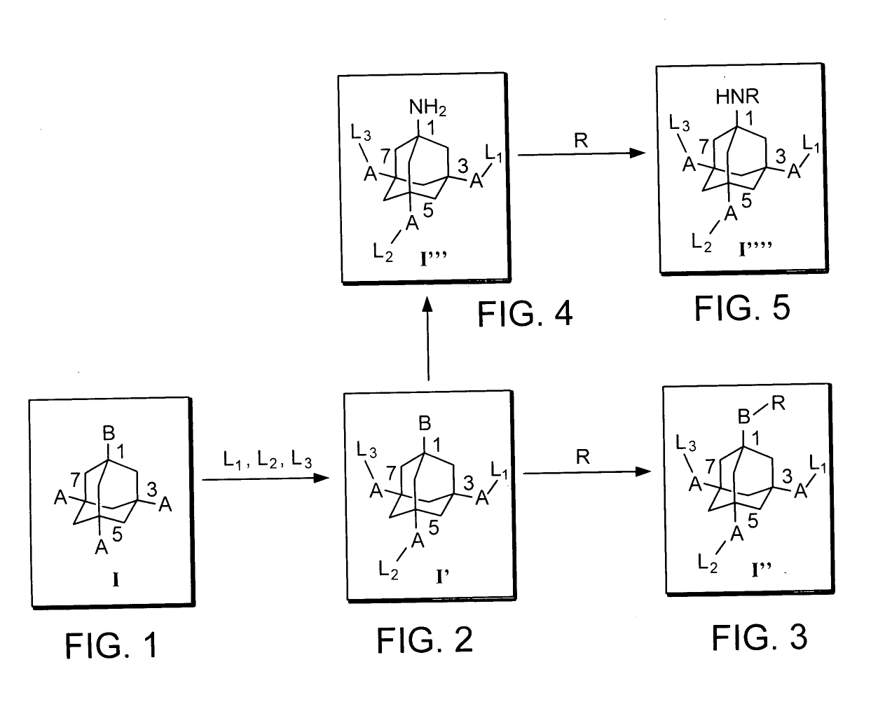

Referring to FIG. 1, compounds of Structure I are provided in which B is H, F, Cl, Br, I, CN, an N-acetyl group, an ammonium group, an amino group, or a protected amino group, and each A is independently a moiety that includes a carboxylic acid group, or an ester, anhydride, or acid halide thereof.

The protected amino group can be protected with, e.g., a t-butoxycarbonyl (Boc) or a benzyloxycarbonyl protecting group.

In some embodiments, each A is (CH₂)ₙCO₂H, where n is between about 0 and about 12 (e.g., 2, 5, 8, or 10). For example, A can be CO₂H (n=0), CH₂CO₂H (n=1), or CH₂CH₂CO₂H (n=2). In some embodiments, an ester, e.g., a N-hydroxysuccinimide ester (NHS), of the carboxylic acid is provided, rather than the acid.

In specific embodiments, B is an N-acetyl group, Br, an ammonium group, e.g., NH₃Cl, or a protected amino group, e.g., protected with a t-butoxycarbonyl (Boc) group, and each A is CH₂CH₂CO₂H.

In specific implementations, B is HN(Boc), and each A is an NHS ester of a carboxylic acid group, e.g., CH₂CH₂CO₂H or CO₂H. In other specific implementations, B is CN, and each A is the methyl ester of CO₂H.

Methods of Making Base Adamantane Derivatives

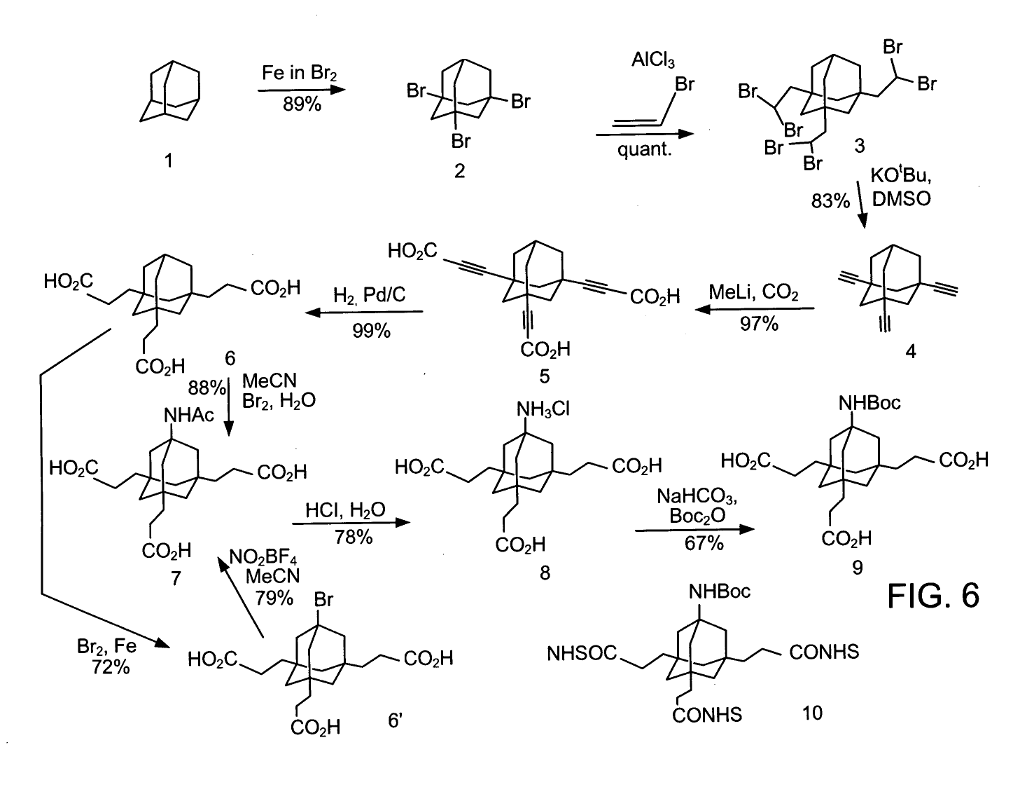

In a specific embodiment, each A is a N-hydroxylsuccinamide ester (NHS) of the CH₂CH₂CO₂H, and B is a t-butoxycarbonyl (Boc) protected amino group. Such an embodiment is represented by compound 10 of FIG. 6. FIG. 6 illustrates one synthetic scheme that can be used to prepare compound 10 from adamantane 1. As shown in FIG. 6, tribromide 2 can be prepared from adamantane 1 by heating adamantane 1 together with Br₂ and Fe for a sufficient time (e.g., 10 to 20, e.g., 15 hours) at reflux. Treating tribromide 2 with vinyl bromide and AlCl₃, results in bromoalkane 3. Treatment of bromoalkane 3 with potassium t-butoxide in DMSO gives triethynyladamantane 4. Triethynyladamantane 4 can be converted to the carboxylic acid form 5 by first forming a lithium acetylide (intermediate not shown), and subsequently quenching with carbon dioxide. Hydrogenation of the alkyne functions gives the saturated, trisubstituted (tricarboxylic acid) adamantane derivative 6.

Tricarboxylic acid adamantane derivative 6 can be converted to the N-acetyl derivative 7 by treatment with Br₂ in acetonitrile/water for 24 hours. Optionally, bromide 6′ can be produced by reaction of 6 with Br₂ and Fe, and then 6′ can be converted to compound 7 via a Ritter reaction of bromide 6′ using nitronium tetrafluoroborate, as described, e.g., by Bach, J. Org. Chem., 44:1739 (1979). The N-acetyl group of compound 7 can be removed by acidic hydrolysis in aqueous HCl, giving ammonium compound 8. Compound 8 can be Boc-protected using Boc₂O, and sodium bicarbonate, yielding compound 9.

Finally, the carboxylic acid groups of the Boc-protected compound 9 can be converted to NHS-ester groups using N-hydroxysuccinimide (NHS) and EDC, 1-ethyl-3-[3-dimethylamino-propyl]carbodiimide hydrochloride, producing compound 10, which can be purified, e.g., by crystallization from isopropanol.

Other methods have been described in Maison et al. Organic Letters, 6(24), 4567 (2004).

Other Adamantane Base Derivatives

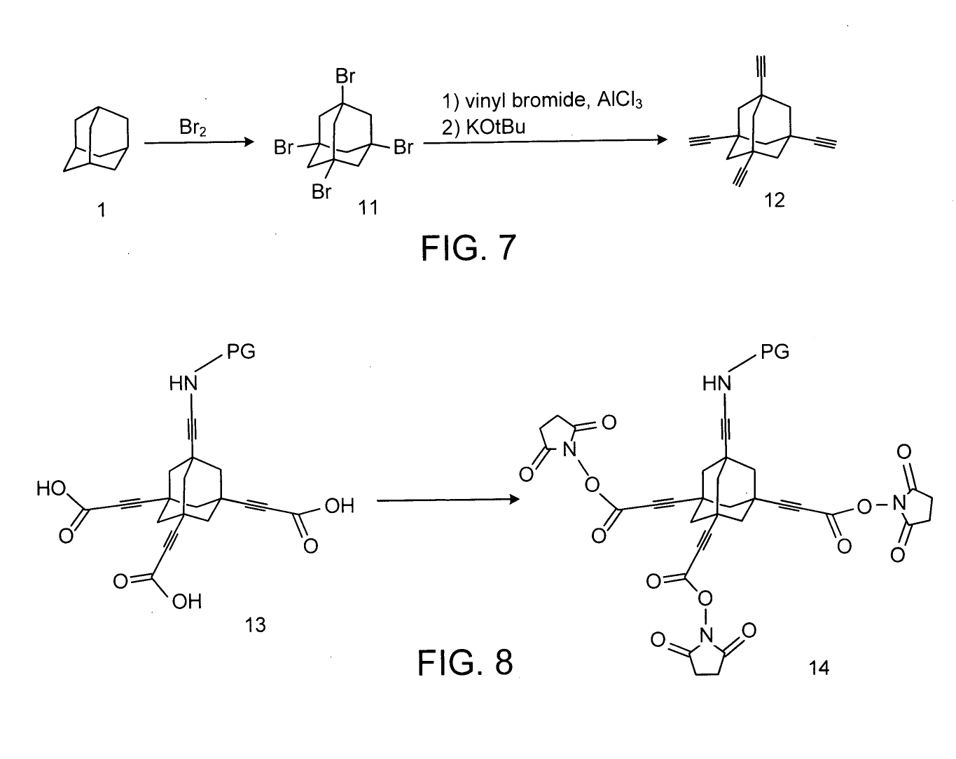

Adamantane derivatives that include two parts can be provided by other approaches. For example, referring to FIG. 7, one synthetic approach starts with adamantane 1, which after bromination gives 1,3,5,7-tetrabromoadamantane 11, as described by Solott in “A Facile Route to 1,3,5,7-Tetraminoadamantane. Synthesis of 1,3,5,7-Tetranitroadamantane,” J. Org. Chem., 1980, 45, 5405. Alkenylation of tetrabromoadamantane 11 with vinyl bromide in the presence of aluminum chloride gives 1,3,5,7-tetra-(dibromomethyl)adamantane (not shown), which after dehydrohalogenation with potassium t-butoxide yields 1,3,5,7-tetraethenyl-adamantane 12.

Referring to FIG. 8, by selective coupling to one of the four ethenyl groups in 12, a protected primary amino functionality can be introduced. Carboxylation of the remaining three ethenyl groups gives the N-protected (PG) carboxylic acid 13. After converting the carboxylic acid groups into NHS esters compound 14 results. In compound 13, not only is the admantane nucleus conformationally rigid, but so are the arms that extend outwardly from the carbons at the 1, 3, 5 and 7 due to hindered rotation about the ethenyl groups, and each carbonyl or amino group conjugated thereto.

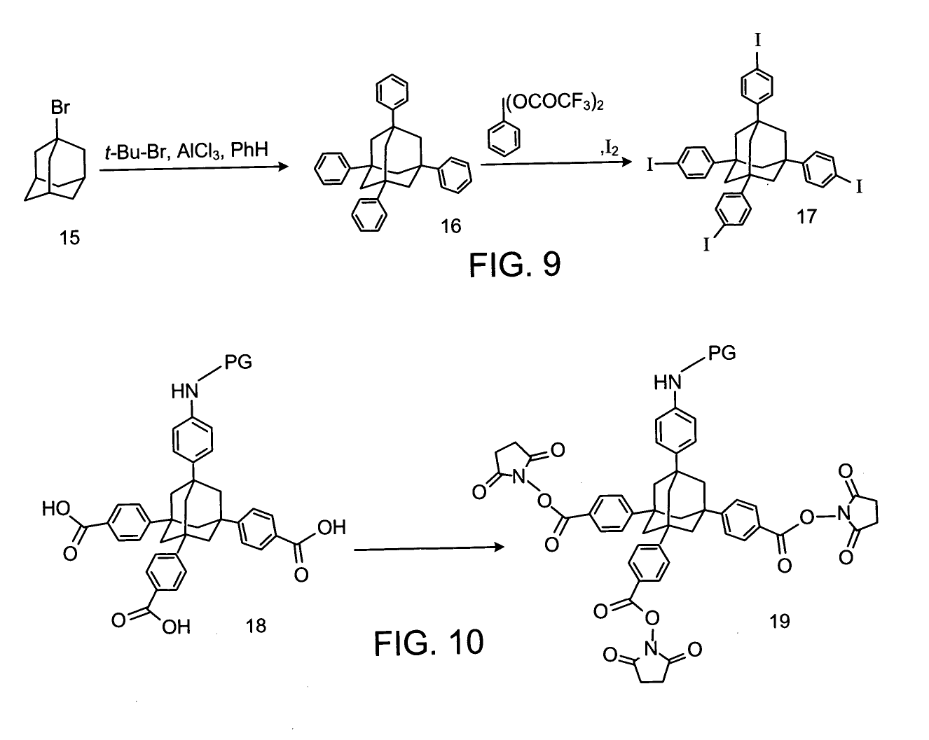

Referring to FIG. 9, another approach can start with a bromoadamantane 15, which can be arylated to give 1,3,5,7-tetraphenyl-adamantane 16 in a mixture of benzene, t-butyl bromide and aluminum chloride. Such a reaction scheme has been described by Aleksey et al. in “Nanoscale 1,3,5,7-Tetrasubstituted Adamantanes and p-Substituted Tetraphenyl-methanes for AFM Applications” Org. Lett., 4(21), 3631 (2002). Consecutive treatment with [bis(trifluoroacetoxy)iodo]benzene and iodine gives the tetraiodide 17.

Referring to FIG. 10, after one of the iodo-substituents in 17 is converted to a protected primary amino group, the remaining iodo substituents can be converted to carboxylic acid groups, producing 18. Compound 18 can, in turn, can be converted to the reactive NHS ester derivative 19. In compound 19, not only is the admantane nucleus conformationally rigid, but so are the arms that extend outwardly from the carbons at the 1, 3, 5 and 7 due to hindered rotation. The NHS ester groups allow conjugation to 1, 2, or 3 different molecules, e.g., targeting ligands, or spacers, e.g., rigid, hydrophilic spacers (discussed further below). After the desired conjugation of the adamantane core and deprotection of the amino group, the free amine can be conjugated to other molecules, e.g., contrast agents or tumor killing agents.

Generally, rigid base admantane derivatives contain rigid moieties attached to bridgehead carbon atoms at 1, 3, 5 and 7 positions. Generally such moieties include functionality, e.g., carboxylic acid groups, an ester, an anhydride, an acid halide, a halogen, an amino group, or hydroxyl group, that enables further functionalizatiion of the base adamantane derivatives. For example, the moieties can exhibit hindered rotation, e.g., due to steric hindrance of proximate groups, constrained ring systems, multiple bonds or conjugation (e.g., an α,β-unsaturated carbonyl). Generally, for enhanced rigidness, the moieties contain less than 12 carbons along their backbone, e.g., less than 8, 6, 5, 4, 3, or less than 2 carbon atoms along their backbone.

Reactions of Adamantane Derivatives

Compounds 4, 5, and 6 of FIG. 6 represent compounds that have functional groups that can be used to further functionalize the adamantane derivatives. Compounds 4 and 5 are generally more rigid than compound 6. For example, compound 4 includes unsaturation that can be used to graft on other functionality, for example, by addition of an acid or halogen across the triple bond. Compound 5, in addition to having unsaturation, has reactive acid groups that can react with nucleophiles, e.g., an amine, an anion, a hydroxyl group, e.g., from a hydroxyl terminated polyether, to produce additional compounds. Such additional grafted-on functionality can be used as spacers to enable the targeting ligands to spaced further apart.

In some embodiments, the linking moiety includes a hydrocarbon chain that includes a terminal carboxylic acid, ester, anhydride, acid halide, halogen, amino group, or hydroxyl group. In other embodiments, the linking moiety includes a substituted hydrocarbon chain that comprises a terminal carboxylic acid, ester, anhydride, acid halide, halogen, amino group, or hydroxyl group.

Referring to FIGS. 1 and 2, compounds of Structure I′ can be prepared from compounds of Structure I by reacting the compound of Structure I, e.g., with NHS-ester groups, with a spacer group (linking group) and/or a ligand, e.g., a targeting ligand, such that the ligand or spacer group reacts selectively with A through the carboxylic acid, ester, anhydride, or acid halide of A. Each A can be the same or different.

For example, compounds of Structure I′ can are formed by reacting compounds of Structure I with a targeting ligand, e.g., a protein, a protein fragment, a peptide, e.g., octreotide (Sandostatin®), a low molecular weight peptide, an antibody, a carbohydrate, or an antigen, having a nucleophilic moiety. The nucleophilic moiety can be, for example, a primary amine group, a thiol group, or a hydroxyl group. Specific proteins, protein fragments, peptides, antibodies, carbohydrates, or antigens useful as targeting ligands are described in “RADIO-LABELED COMPOUNDS, COMPOSITIONS, AND METHODS OF MAKING THE SAME,” U.S. Ser. No. 11/156,259, filed on Jun. 17, 2005. Also see Frangioni, “MODIFIED PSMA LIGANDS AND USES RELATED THERETO”, WO 02/098885, filed on Feb. 7, 2002.

A specific targeting ligand is the RGD peptide, which specifically binds to alphₐᵥβ₃ integrin. It is known that this integrin is overexpressed by various tumors, and thus, these RGD targeting peptides enable the adamantine derivatives to preferentially label tumors that overexpress these integrins.

Other targeting ligands include melanocyte stimulating hormone (MSH), which targets melanoma cells, or bombesin, somatostatin, or Sandostatin™ (synthetic), which target somatostatin receptors.

In some embodiments, a rigid spacer group, e.g., a rigid, hydrophilic spacer group is used such that a rigid molecule results. Examples of rigid spacer groups include molecules existing as α-helices, or rigid natural or synthetic polymers having reduced mobility along their backbone. For extra rigidity, the α-helices can be cross-linked, e.g., through peptide linkages or sulfur-sulfur bonds. Grubbs has described other cross-linking methods using metals, e.g., in Journal Organic Chemistry, 66(16), 5291 (2001). Such α-helices have been described by Fairlie in Journal American Chemical Society, 126(46), 15096 (2004); Journal Mol. Graph Model, 21(5), 341 (2003); Journal American Chemical Society, 127(18), 6563 (2005); and Journal American Chemical Society, 127(9), 2974 (2005). The described α-helices become covalently bound to desired locations of the admantane nucleus by reaction, e.g., of an amino group of the α-helices with an electrophile, e.g., a carboxylic acid group, at bridgehead locations. Generally, the spacers include functional groups, e.g., an amino group, on the terminal end of the molecule away from the adamantane nucleus that enables even further functionalization, e.g., functionalization with any of the targeting ligands described herein.

Other rigid spacer molecules include, e.g., dimeric, coiled coils, e.g., tropomyosin, which are optionally cross-linked, such as those described by Hodges in Journal Biological Chemistry, 279(20), 21576 (2004); Protein Science, 13(3), 714 (2004); Journal Biological Chemistry, 278(37), 35248 (2003); Journal of Molecular Recognition, 16(1), 37 (2003); Journal Chromatogr. A., 972(1), 101 (2002), Journal Cell Biochemistry, 83(1), 99 (2002); Journal Cell Biochemistry, 83(1) 33 (2001); Circ. Res., 82(2), 261 (1998); Journal Molecular Biology, 271(5), 728 (1997); and Journal Biological Chemistry, 272 (16), 10529 (1997).

The length of the spacer is, e.g., from about 1 nm to about 100 nm, e.g., from about 5 or 10 nm to about 75 nm, or from about 7.5 nm to about 50 nm.

In some embodiments, the molecular weight of the rigid spacer is from about 300 Daltons to about 300,000 Daltons, e.g., from about 750 Daltons to about 150,000 Daltons, or from about 2,000 Daltons to about 100,000 Daltons. Higher molecular weight spacers can increase blood half life by delaying renal clearance.

Without wishing to be bound by any particular theory, it is believed that targeting groups at terminal ends of rigid arms can bind with greater efficiency to complementary sites on cells because the arms have lower conformational entropy. Polyvalent interactions in biological systems have been described by Whitesides in Angew. Chem. Int. Ed., 37, 2754 (1998). Also without wishing to be bound by any particular theory, it is believed that making the rigid arms relatively hydrophilic enables targeting groups at terminal ends of the rigid arms to bind with greater efficiency to complementary sites on cells, because cells are surrounded by a dense hydrophilic glycocalyx.

Referring particularly to FIGS. 2 and 3, compounds of Structure I″ can formed by reacting compounds of Structure I′ with a reporter group or therapeutic group such that the reporter or therapeutic group reacts selectively with B.

Referring to FIGS. 2 and 4, in some embodiments, B includes a protecting group, e.g., a t-butoxycarbonyl (Boc) or benzyloxycarbonyl protecting group. In such embodiments, B can be deprotected, e.g., with an acid, exposing an amino group. Referring now to FIGS. 4 and 5, the exposed amino group can be reacted with a reporter or therapeutic group or molecule.

In general, reporter or therapeutic molecules contain an electrophilic group, e.g., a carboxylic acid group, an ester group, e.g., an NHS group, or an acid chloride group. For example, the NHS ester of the NIR fluorophore CW800 (LI-COR, Lincoln, Nebr.) can be used to create optical imaging probes. The NHS ester of the compound DOTA (Macrocyclics, Dallas, Tex.) can be used to chelate gadolinium for MRI imaging or to chelate indium-111 for radio-scintigraphic imaging. Also, for example, the NHS ester of the compound MAS₃ can be used to chelate technetium-99m for SPECT imaging.

Therapeutic molecules include chemotherapeutic agents. Examples include NHS ester derivatives of doxorubicin, or NHS ester derivatives of Taxol. The DOTA molecule can also be used, when loaded with a beta-emitting radioisotope for use in radiotherapy.

Applications and Administration

Generally, the adamantane derivatives described herein can be used in medicine, e.g., as medical imaging agents when appropriately conjugated, as drugs, or as drug delivery systems. Particularly useful for imaging are those derivatives that are conjugated with ligands, linking molecules, and/or reporter molecules.

For example, the new derivatives can be used for imaging a tissue or portion of a body, by selecting a targeting ligand that binds selectively to a moiety in the tissue to be imaged, and linking a reporter group to the derivative. The labeled derivative is then administered to the tissue in an amount effective an by a route effective to deliver the derivative to the target tissue. The tissue is then imaged after a time sufficient for the targeting ligand to selectively bind to the moiety in the tissue. The imaging modality used corresponds to the reporter group used to label the derivative.

Various imaging modalities can be used, and the derivatives can be labeled with different reporter groups, so that the same derivative can be used to image the same tissue using two or more different imaging modalities. For example, optical, MRI, PET, and SPECT imaging can all be used.

For example, ¹⁸F radio-labeled conjugates can be prepared by reacting compounds of Structure I with ligands that have a specific affinity for certain abnormal cells, e.g., cancer cells, at the A positions and an ¹⁸F label at the B position. Such derivatives can be useful, e.g., for in vivo pathology imaging, e.g., tumor imaging using PET. When properly configured, e.g., when the ligands, linking molecules, or reporter molecules include a molecular architecture that can bind specifically to a moiety of interest, the ¹⁸F radio-labeled conjugates can be used to specifically image abnormalities of the bladder, the brain, kidneys, lungs, skin, pancreas, intestines, uterus, adrenal gland, and eyes, e.g., retina.

In another example, a near-infrared reporter molecule (i.e., a contrast agent), e.g., CW800-NHS (LI-COR, Lincoln, Nebr.), is attached to the deprotected amine of FIG. 4, to create optical imaging probes. In addition, DOTA derivatives can be used for either MRI or radio-scintigraphy.

Any known imaging or therapeutic agents that can be linked to B in the new adamanane derivates can be used.

The adamantane derivatives can be incorporated into pharmaceutical compositions for administration for therapeutic use or subsequent imaging. Such compositions typically include the adamantane derivative and a pharmaceutically acceptable carrier. As used herein a “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include oral or parenteral, e.g., intravenous, intradermal, subcutaneous, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, e.g., tromethamine; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and should be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. Prevention of the action of microorganisms can be achieved by various antibacterial and antifungal agents, e.g., parabens, chlorobutanol, phenol, ascorbic acid, thimerosal, and the like. In many cases, it will be desirable to include isotonic agents, e.g., sugars, polyalcohols such as mannitol, sorbitol, or sodium chloride in the composition. Prolonged absorption of the injectable compositions can be brought about by including in the composition an agent which delays absorption, for example, aluminum monostearate, and gelatin.

Sterile injectable solutions can be prepared by incorporating the adamantane derivatives in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Such compositions can also be compounded to minimize exposure to gastric enzymes or to facilitate uptake by the intestinal tract.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents and liposomes. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery. Such preparations are particularly useful for treating conditions associated with pathogen invasion of the lower intestinal tract.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, e.g., as described in U.S. Pat. No. 4,522,811.

Oral or parenteral compositions can be provided in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of pharmaceutical compositions containing one or more of the new adamantane derivatives can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in to minimize potential damage to non-target cells (e.g., cells that are not undergoing an undesirable inflammatory reaction) and, thereby, reduce side effects. In general, the new adamantane derivatives described herein should be well tolerated by an animal (e.g., mouse, non-human primate, or human).

The data obtained from the cell culture assays and animal studies can be used in formulating a range of dosage for use in humans. The dosage of such compounds lies generally within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models (e.g., of infection or inflammatory disease) to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography or ELISA.

The pharmaceutical compositions can be included in a container, pack, or dispenser together with instructions for administration.

### EXAMPLES

The invention is further described in the following examples, which do not limit the scope of the invention described in the claims.

General

Melting points (or decomposition points) were determined in open capillaries, and are uncorrected. ¹H-NMR and ¹³C-NMR spectra were recorded on either a Bruker-Karlsruhe AMX 400 spectrometer (400 MHz/100.6 MHz), or a Bruker-Karlsruhe DRX 5001 spectrometer (500 MHz/125.8 MHz). Chemical shifts (6) are presented in parts per million (ppm) and coupling constants (J) are presented in hertz (Hz). Tetramethylsilane was used as an internal standard (TMS, 0 ppm). Mass spectra were obtained with either a Varian MS MAT 311A in EI mode, a VG/70-250 F (VG Analytical) instrument in FAB mode (p-nitrobenzyl alcohol matrix), or on a MAT 95 Trap XL (Thermo Finnigan) instrument in ESI mode (positive mode). Polypropylene glycol or polyethylene glycol was used as internal standard for the MAT 95 instrument. Compounds 2 and 4 were synthesized according to procedures set forth by Delimarskii in Ukrainskii Khimicheskii Zhurnal (Russian Edition) 1988, 54, 437, and Malik in J. Polym. Sci., Part A: Polym. Chem. 1992, 30, 1747, respectively.

### Example 1

### Synthesis of Compound 5

To a solution of 9.40 g alkyne 4 (45.2 mmol) in 500 ml dry THF was added dropwise 84.6 ml of a 1.6 M MeLi solution in THF (135 mmol) at 0° C. under a nitrogen atmosphere. After addition was complete, the resulting suspension was stirred for an additional 30 min at 0° C., cooled to −70° C., and then a stream of CO₂ gas was passed through the suspension for 10 min. The reaction mixture was warmed to room temperature and poured on 1000 ml ice water. This alkaline solution was washed two times with each 200 ml diethylether and was then acidified to pH˜1 with concentrated HCl. The resulting suspension was extracted three times with 200 ml dichloromethane. The combined extracts were dried over Na₂SO₄, and the solvent was removed in vacuo to give 14.92 g of 5 (97% yield). mp: 229-230° C. ¹H-NMR (DMSO-d₆, 400 MHz): δ 1.79 (d, 6H, J=1.8), 1.92 (d, 3H, J=12.2), 2.00 (d, 3H, J=12.2), 2.11-2.13 (m, 1H), 13.42 (br, 3H). ¹³C-NMR (DMSO-d₆, 100 MHz): δ 26.8, 29.3, 38.2, 42.9, 74.0, 91.1, 154.2. MS (EI): m/z [%]=296 [8], 252 [55], 208 [100].

### Example 2

### Synthesis of Compound 6

A solution of 7.92 g of alkyne 5 (23 mmol) in 200 ml THF was treated with 50 mg of 10% Pd on charcoal with stirring under a hydrogen atmosphere for 78 h. The reaction mixture was filtered through a pad of celite and the solvent removed in vacuo to give 8.12 g of a colourless solid that was purified by re-crystallization from acetonitrile to give 8.02 g of the hydrogenated carboxylic acid 6 (99% yield). Compound 6 decomposes at 107° C. ¹H-NMR (D₂O, 500 MHz): δ 1.00 (d, 3H, J=11.7), 1.08 (d, 3H, J=11.7), 1.25 (d, 6H, J=1.9), 1.29-1.32 (m, 6H), 1.97-1.99 (m, 1H), 2.04-2.07 (m, 6H). ³C-NMR (D₂O, 100 MHz): δ 29.5, 32.0, 33.4, 40.3, 40.9, 46.1, 185.4. MS (EI): m/z [%]=352 (MH⁺) [2], 334 [20], 316 [35], 279 [70], 261 [100].

### Example 3

### Synthesis of Compound 6′

0.39 g iron powder (7.1 mmol) was added at 0° C. to 5 ml bromine. The resulting mixture was stirred for 30 min at 0° C., and then 0.50 g of carboxylic acid 6 (made in Example 2) was added. The reaction mixture was stirred at 5° C. for 12 h, and then poured into an ice/HCl mixture. The resulting suspension was treated with Na₂SO₃ to destroy any remaining bromine, and was extracted 3 times with 100 ml ethyl acetate. The combined organic layers were washed with diluted HCl, dried over Na₂SO₄. The solvent was removed in vacuo to give 735 mg of a yellow solid as a crude product. This solid was stirred for 10 minutes with hot dichloromethane, and filtered to give 441 mg pure bromide 6′ as a colourless solid (72% yield). Compound 6′ decomposes at 190° C. ¹H-NMR (DMSO-d₆, 400 MHz): δ 1.07 (d, 3H, J=12.2), 1.16 (d, 3H, J=12.2), 1.38-1.42 (m, 6H), 1.91 (s, 6H), 2.14-2.18 (m, 6H). ¹³C-NMR (DMSO-d₆, 100 MHz): δ 27.7, 36.9, 37.5, 43.4, 52.0, 174.9.

### Example 4

### Synthesis of Compound 7 from Compound 6′

To a solution of 100 mg bromide 6′ (0.23 mmol) in 5 ml dry acetonitrile was added NO₂BF₄ (0.46 mmol), and the resulting yellow solution was stirred for 48 h under a nitrogen atmosphere at room temperature. Water (10 ml) was added, and the pH was adjusted to 10 with 2N NaOH. The aqueous solution was washed two times with 20 ml dichloromethane, acidified with 4 N HCl to pH 1, and then extracted three times with 50 ml ethyl acetate. After drying the combined organic layers over Na₂SO₄, filtration and evaporation of the solvent gave 90 mg crude 7.

### Example 5

### Synthesis of Compound 7 from Compound 6

To a solution of 881 mg carboxylic acid 6 (2.5 mmol) in a mixture of 390 mg acetonitrile (9.5 mmol) and 149 mg water (8.3 mmol) was added 8.39 g dry bromine (52.5 mmol). The resulting solution was heated to reflux for 15 h. After cooling to room temperature, the reaction mixture was poured into 100 ml of acidic ice water (pH=1), and treated with Na₂SO₃ to destroy any remaining bromine. Extraction with three times with 150 ml of ethyl acetate, drying over Na₂SO₄, and evaporation of the solvent in vacuo gave 1.06 g crude product. The crude product was re-crystallized to give 0.90 g N-acetylated amino acid 7 (88% yield). ¹H-NMR (DMSO-d₆, 400 MHz): δ 0.99 (d, 3H, J=12.1), 1.04 (d, 3H, J=12.1), 1.35-1.39 (m, 6H), 1.50 (s, 6H), 1.72 (s, 3H), 2.11-2.15 (m, 6H).

### Example 6

### Synthesis of Compound 8

A suspension of the N-acetylated amino acid 7 in a mixture of 28 ml water and 3.6 ml concentrated HCl was heated to reflux for 24 h. After cooling to room temperature the acidic solution was washed two times with 20 ml ethyl acetate. Water was removed in vacuo to give 693 mg of amino acid 8 as a colourless solid (78% yield). ¹H-NMR (D₂O, 500 MHz): δ 1.21-1.27 (m, 6H), 1.56 (s, 6H), 1.59-1.62 (m, 6H), 2.39-2.43 (m, 6H). ¹³C-NMR (D₂O, 100 MHz): δ 28.2, 35.1, 36.8, 43.5, 43.6, 54.3, 179.6.

### Example 7

### Synthesis of Boc-Protected Compound 9

To a solution of 560 mg acid 8 (1.39 mmol) in 16 ml dioxane/water (1:1) was added 584 mg NaHCO₃ (6.95 mmol) and 437 mg Boc₂O (2 mmol) in 3 ml dioxane. The solution was stirred for 48 h at room temperature. A second portion of 0.30 g Boc₂O (1.39 mmol) together with 0.12 g NaHCO₃ (1.39 mmol) was added, and the solution was stirred for an additional 12 h at room temperature. Water (50 ml) was added, and the solution was washed two times with 25 ml ethyl acetate. The alkaline aqueous phase was acidified with 2N HCl to pH 2, and extracted three times with 50 ml ethyl acetate. Drying of the combined organic layers over Na₂SO₄, followed by evaporation of the solvent gave 365 mg of the Boc-protected amino acid 9 as a colorless foam (56% yield). ¹H-NMR (DMSO-d₆, 500 MHz): δ 0.97-1.03 (m, 6H), 1.36 (s, 9H), 1.36-1.39 (m, 6H), 1.43 (s, 6H), 2.11-2.15 (m, 6H), 6.38 (br, 1H), 11.98 (br, 3H). ¹³C-NMR (DMSO-d₆, 100 MHz): δ 27.9, 28.3, 34.3, 37.5, 44.6, 51.7, 175.

### Example 8

### Synthesis of Compound 10

To a solution of the 300 mg Boc-protected acid 9 (0.64 mmol) and 221 mg N-hydroxysuccinimide (1.92 mmol) in 5 ml dry THF was added 402 mg dicyclohexyl-carbodiimide in 5 ml dry THF at 0° C. The solution was stirred for 12 h at 5° C., and then filtered and the solvent removed in vacuo. The remaining solid was dissolved in dichloromethane, filtered, and then the solvent removed in vacuo to give a crude product that was re-crystallized from 2-propanol. Re-crystallization yielded 403 mg of the NHS-ester 10 as a colorless solid (83%). ¹H-NMR (CDCl₃, 400 MHz): δ 1.13 (d, 3H, J=12.2), 1.22 (d, 3H, J=12.2), 1.43 (s, 9H), 1.61 (s, 6H), 1.67-1.71 (m, 6H), 2.58-2.62 (m, 6H), 2.82-2.87 (m, 14H), 4.61 (br, 1H). ¹³C-NMR (CDCl₃, 100 MHz): δ 21.9, 25.3, 25.7, 28.6, 35.1, 36.7, 44.8, 44.9, 52.3, 169.26, 169.29.

### OTHER EMBODIMENTS

A number of embodiments of the invention have been described. Nevertheless, it will be understood that various modifications may be made without departing from the spirit and scope of the invention.

We claim:

1. A compound comprising an adamantane core having four bridgehead positions, wherein three of the four bridgehead positions are functionalized with a first moiety that comprises a carboxylic acid group, or an ester, anhydride, or acid halide thereof; wherein the fourth bridgehead position comprises a second moiety different from the first moiety; and wherein the second moiety comprises a nucleophile or a protected nucleophile.

2. The compound of claim 1, wherein the protecting group is t-butoxycarbonyl (Boc) or benzyloxycarbonyl.

3. The compound of claim 1, wherein the nucleophile is an amino group.

4. The compound of claim 1, further comprising a ligand bound to at least a portion of at least one of the first moieties.

5. The compound of claim 1, further comprising a reporter molecule bound to at least a portion of the second moiety.

6. A compound of Structure I 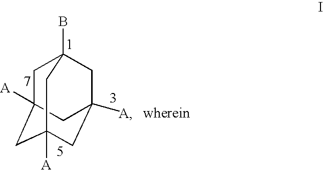
B is H, F, Cl, Br, I, CN, an N-acetyl group, an ammonium group, an amino group, or a protected amino group; and
each A is independently a moiety that comprises a carboxylic acid group, or an ester, anhydride, or acid halide thereof.

7. The compound of claim 6, wherein B is an N-acetyl group, Br, NH₃Cl or HN(Boc), and wherein each A is CH₂CH₂CO₂H.

8. The compound of claim 6, wherein the protecting group is t-butoxycarbonyl (Boc) or benzyloxycarbonyl.

9. The compound of claim 6, wherein B is HN(Boc), and wherein each A is an NHS ester of CH₂CH₂CO₂H.

10. The compounds of claim 6, wherein B is a protected amino group, and wherein each A is an NHS ester of COOH.

11. The compound of claim 6, wherein B is CN, and wherein each A is a methyl ester of COOH.

12. The compound of claim 6, wherein at least one A includes a linking moiety.

13. The compounds of claim 12, wherein the linking moiety comprises a hydrocarbon chain that comprises a terminal carboxylic acid, ester, anhydride, acid halide, halogen, amino group, or hydroxyl group.

14. The compound of claim 6, further comprising a ligand bound to at least a portion of each A, wherein each ligand is the same or different than one or more other ligands.

15. The compound of claim 14, wherein the ligand comprises a targeting compound.

16. The compound of claim 15, wherein the targeting compound is an RGD peptide, a melanocyte stimulating hormone (MSH), or a somatostatin.

17. The compound of claim 15, wherein the compound is further linked to a contrast agent.

18. The compound of claim 6, further comprising a reporter molecule bound to at least a portion of B.

19. The compound of claim 6, further comprising a therapeutic agent bound to at least a portion of B.

20. The compound of claim 19, wherein the therapeutic agent is selected from the group consisting of doxorubicin, taxol, DOTA, a moiety comprising boron, and a pro-apoptotic peptide.

21. A compound comprising an adamantane core having a molecular arm extending from each of four bridgehead positions, wherein at least one of the molecular arms includes a spacer portion, the spacer portion comprising a carboxylic acid group, or an ester, anhydride, or acid halide thereof.

22. The compound of claim 21, wherein at least one molecular arm comprises a nucleophile or a protected nucleophile.

23. The compound of claim 21, wherein the spacer portion comprises an α-helix or a dimeric coil-coil helix.

24. The compound of claim 23, wherein the α-helix or coil-coil helix is cross-linked.

25. The compound of claim 21, wherein the spacer portion has a molecular weight of from about 500 Daltons to about 300,000 Daltons.

26. The compound of claim 21, wherein the spacer portion has a length of from about 1 nm to about 100 nm.

27. The compound of claim 21, wherein the at least one arm that includes the spacer portion, further includes a targeting moiety bonded to the spacer portion.

28. The compound of claim 21, wherein at least three molecular arms include spacer portions.

29. The compound of claim 21, wherein three molecular arms include spacer portions, and wherein the fourth arm comprises a nucleophile or a protected nucleophile.
